(19) United States
(12) Patent Application Publication
(10) Pub. No.: US 2009/0156964 A1
(43) Pub. Date: Jun. 18, 2009

(54) Flip-Top Integrated-Diagnostic Instrument

(76) Inventor: John P. Creaven, Granger, IN (US)

Correspondence Address: NIXON PEABODY LLP, 161 N. CLARK STREET, 48TH FLOOR, CHICAGO, IL, 60601, US

(21) Appl. No.: 11/989,365

(22) PCT Filed: Sep. 11, 2006
(86) PCT No.: PCT/US2006/035141

§ 371 Date: Jan. 24, 2008

Related U.S. Application Data
(60) Provisional application No. 60/720,841, filed on Sep. 27, 2005.

(51) Int. Cl. A61B 5/151

(52) U.S. Cl. 600/583

(57) ABSTRACT

An integrated-diagnostic instrument adapted to determine an analyte concentration of a fluid sample using a test sensor is disclosed. The instrument comprises a first portion including at least one opening formed therein. The opening is adapted to receive a test sensor. The first portion forms at least one compartment adapted to receive a lancing device and includes a user-interface mechanism. The instrument further comprises a second portion being hingedly connected to the first portion. The second portion includes a display.

### FIELD OF THE INVENTION

The present invention generally relates to an integrated-diagnostic instrument and, more particularly, to a flip-top integrated-diagnostic instrument that is used for storing analyte-testing instruments and in determining an analyte concentration (e.g., glucose) in a fluid (e.g., blood).

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests can be used to determine what, if any, insulin or other medication needs to be administered. In one type of testing system, test sensors are used to test a fluid such as a sample of blood.

Many individuals perform testing of their blood glucose at several different locations. These locations often include their home or place of employment, such as an office building or work site. Many of these individuals who must test for glucose or other analytes carry with them a meter, a container of test sensors, a lancet, disposable lancets, and/or other analyte-testing instruments. The analyte-testing instruments may shift and potentially become damaged while being transported or carried. Transporting the analyte-testing instruments may also have other disadvantages, such as bulkiness and/or inconvenience.

It would be desirable to have an integrated-diagnostic instrument that assists in addressing one or more of the above disadvantages.

### SUMMARY OF THE INVENTION

According to one embodiment of the present invention, an integrated-diagnostic instrument adapted to determine an analyte concentration of a fluid sample using a test sensor is disclosed. The instrument comprises a first portion including at least one opening formed therein. The opening is adapted to receive a test sensor. The first portion forms at least one compartment adapted to receive a lancing device and includes a user-interface mechanism. The instrument further comprises a second portion being hingedly connected to the first portion. The second portion includes a display.

According to another embodiment of the present invention, an integrated-diagnostic instrument adapted to determine an analyte concentration of a fluid sample using a test sensor is disclosed. The instrument comprises a first portion including a user-interface mechanism. The first portion includes at least one opening formed therein. The opening is adapted to receive a test sensor. The first portion forms a plurality of compartments. A first compartment includes a plurality of test sensors. A second compartment includes a lancing device. The instrument further comprises a second portion being hingedly connected to the first portion. The second portion includes a display.

The above summary of the present invention is not intended to represent each embodiment or every aspect of the present invention. Additional features and benefits of the present invention are apparent from the detailed description and figures set forth below.

### BRIEF DESCRIPTION OF THE DRAWINGS

FIG. 1a is a front perspective view of an integrated-diagnostic instrument in an open position according to one embodiment of the present invention.

FIG. 1b is a front perspective view of the integrated-diagnostic instrument of FIG. 1a in a closed position.

FIG. 2a is a top view of a lancing device according to one embodiment.

FIG. 2b is a bottom view of the lancing device of FIG. 2a.

FIG. 3a is a front perspective view of an integrated-diagnostic instrument according to another embodiment of the present invention.

FIG. 3b is a front perspective view of the integrated-diagnostic instrument of FIG. 3a after a lancet endcap has been removed.

FIG. 4a is a perspective internal view of a test-sensor cartridge according to one embodiment.

FIG. 4b is a top view of the cartridge of FIG. 4a.

### DETAILED DESCRIPTION OF THE ILLUSTRATED EMBODIMENTS

The present invention is directed toward a flip-top integrated-diagnostic instrument that determines an analyte concentration in a fluid.

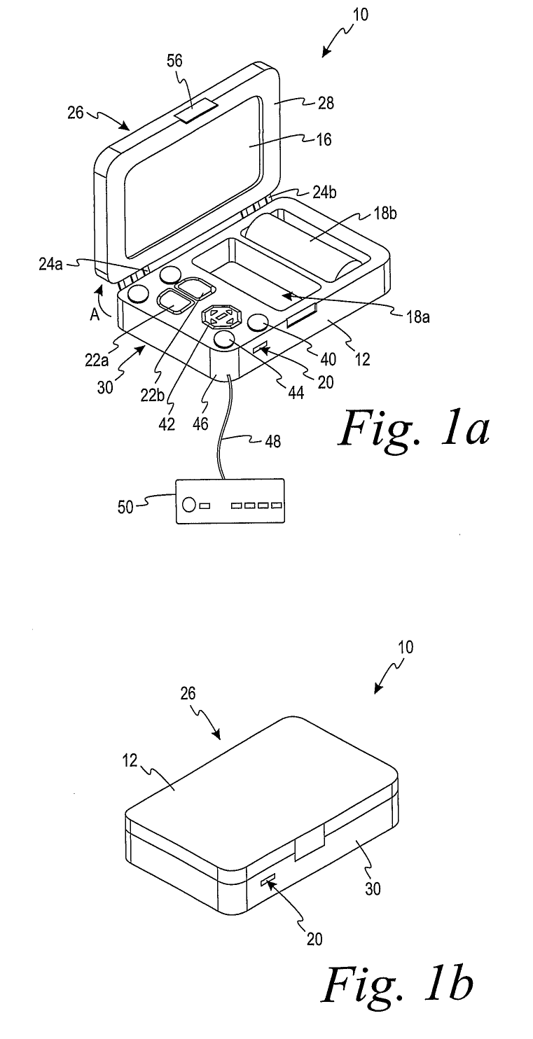

FIGS. 1a,b and 3a,b depict respective integrated-diagnostic instruments according to embodiments of the present invention. The instrument is used to determine concentrations of analytes. Analytes that may be measured using the present invention include glucose, lipid profiles (e.g., cholesterol, triglycerides, LDL, and HDL), microalbumin, hemoglobin A_{1C}, fructose, lactate, and/or bilirubin. The present invention is not limited, however, to these specific analytes, and it is contemplated that other analyte concentrations may be determined. The analytes may be in, for example, a whole blood sample, a blood serum sample, a blood plasma sample, or other body fluids like ISF (interstitial fluid) and/or urine. One non-limiting example of the instrument's use is to determine the glucose concentration in a user's blood or plasma.

Referring to FIGS. 1a,b, an integrated-diagnostic instrument 10 may be powered by a mains power supply, a battery, or any other suitable power source. The mains power supply may include internally operated AC and/or DC power supplies. It may be desirable that the instrument 10 be powered by battery due to the portable nature of the instrument 10.

The integrated-diagnostic instrument 10 comprises a housing 12, a display 16, and at least one compartment 18a,b. The housing 12 includes a first portion 30 and a second portion 26. The first portion 30 forms at least one test-sensor opening 20 therein. The opening 20 is adapted to receive and/or hold a test sensor and assist in determining the analyte concentration of a fluid sample. The housing 12 of the cartridge is typically made of a polymeric material. Non-limiting examples of polymeric materials include polycarbonate, ABS, nylon, polypropylene, and/or combinations thereof. The instrument 10 is desirably sized so that it may fit generally within a user's purse or pocket.

To communicate at least the analyte concentration to the user, the instrument 10 includes a display 16. One example of a display 16 that may be used in the instrument 10 is a liquid-crystal display. The liquid-crystal display typically shows information from the testing procedure and/or in response to signals input by a user-interface mechanism (e.g., buttons 22a, 22b) on the instrument 10. For example, the user-interface mechanism may be depressed to recall and view results of prior testing procedures on the display 16. It is contemplated that other user-interface mechanisms may be used, including, but not limited to, scroll wheels or the like.

According to the present invention, the instrument 10 has a “flip-top” feature, wherein an end of the second portion 26 is hingedly connected to an end of the first portion 30 via a hinged mechanism. The hinged mechanism allows for the second portion 26 to be moved between an open position (see FIG. 1a) and a closed position (see FIG. 1b). In the illustrated embodiments, the hinged mechanism includes a pair of hinges 24a,b. The display 16 is electrically connected to the first portion 30 by a flexible circuit, which runs generally through the hinges 24a,b. To move the second portion 26 between an open position and a closed position, the second portion 26 is rotated in a first direction about the hinges 24a,b, as shown by Arrow A in FIG. 1a. Although in the embodiment shown in FIG. 1a, the hinged mechanism comprises a pair of hinges 24a,b, in other embodiments, a single hinge, such as a continuous or piano hinge, running along a part of or the entire length of the second portion 26 and the first portion 30 may be employed.

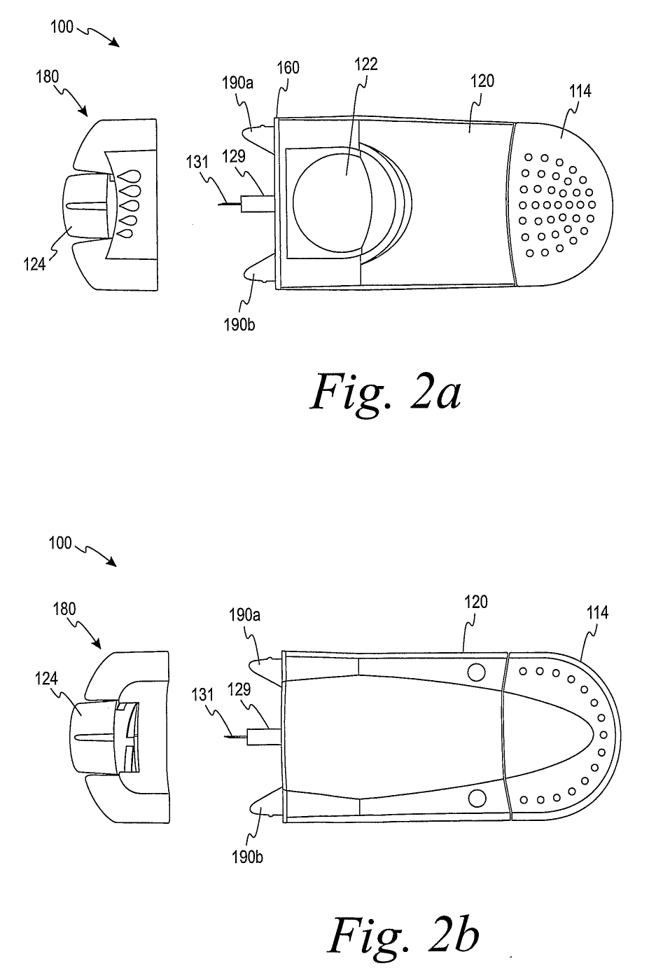

To enhance storage capabilities and convenience to a user, the instrument also includes at least one compartment for storing analyte-testing instruments such as, for example, glucose-testing instruments, required for use with the instrument. The instrument 10 desirably includes a plurality of compartments 18a, 18b, as shown in FIG. 1a, for storing the glucose-testing instruments. The glucose-testing instruments may include, but are not limited to, lancets, a lancing device, test sensors, and/or tissues. For example, in FIG. 1a, a compartment 18a may contain unused lancets or a lancing device, such as a lancing device 100 shown in FIGS. 2a-b. Another compartment 18b may contain individually unused test sensors or an unused cartridge that contains a plurality of unused test sensors. It is contemplated that the instrument 10 may include other compartments, for example, to store individually used test sensors or a used cartridge that contains a plurality of used test sensors, a plurality of tissues for cleaning, or other items. Thus, the instrument of the present invention may generally contain substantially all or all of the items used for glucose testing such that the instrument can be a “one-stop” instrument to the user.

An additional benefit of the flip-top feature of the instrument 10 is that the display 16 is generally protected from damage and scratching when the instrument 10 is in the closed position of FIG. 1b while being, for example, transported, carried, or stored. According to the present invention, the display 16 is located on an interior surface 28 of the second portion 26. Thus, when the instrument 10 is in the closed position (see FIG. 1b), the display 16 is located within the instrument 10 and is protected when the instrument is transported, carried, or stored. The instrument 10 may include a releasably lockable mechanism for maintaining the instrument 10 in the closed position. The instrument may further include a release button that may unlock the releasably lockable mechanism. It is contemplated that the second portion 26 may be releasably locked to the first portion 30 of the instrument 10 by at least one latch 56 or any other mechanism suitable for keeping at least a portion of the second portion 26 substantially flush with the first portion 30.

It is desirable that the instrument 10 have a footprint area of less than 12 in² to enhance portability. The instrument 10 may even have a footprint area of about 6 in². The footprint area is the length L of the instrument times the width W of the instrument (see FIG. 1a).

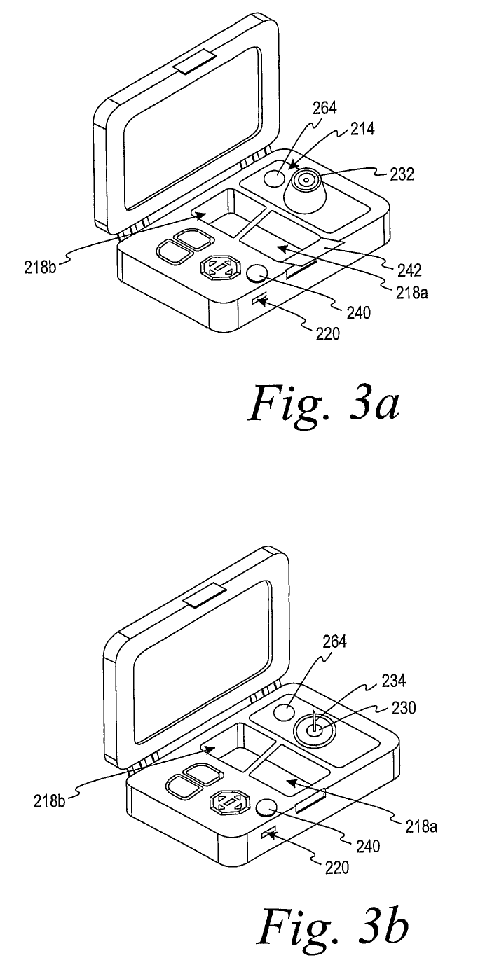

The instrument 10 also serves as a convenient device for carrying and protecting the glucose or other analyte-testing instruments. When in the closed position of FIG. 1b, the flip-top instrument 10 holds the necessary glucose-testing instruments within their respective compartments 18a,b of the housing 12. By holding the glucose-testing instruments in place, the risk of the glucose-testing instruments shifting and/or becoming damaged is reduced and/or eliminated. The compartments may further include a detachable cover or lid 242 (see FIG. 3a). The cover or lid may be desirable to protect the display 16 from damage or scratches potentially caused by the glucose-testing instruments stored within the compartments 18a,b.

Because the display 16 is positioned on the second portion 26, while the at least one compartment 18 and the user-interface buttons 22a,b are positioned on the first portion 30, the display 16 may generally span the length L and width W of the instrument 10. Thus, the display 16 may be relatively large, assisting in the readability of the information displayed on the display 16, especially for those individuals with poor vision. Moreover, a larger display 16 may also be used to display additional information including, but not limited to, graphical interpretations of historical glucose readings. For example, the display 16 typically has an area of at least about 3.75 in², such as 2.5 inch×1.5 inch. The display 16 may even have an area of at least about 8.75 in², such as 3.5 inch×2.5 inch. Some of the information that may be shown on the display may include, but is not limited to, the following: a numerical display, an indication of the number of sensors remaining, an indication to load a cartridge or test sensor into the instrument, an apply-blood indication, a temperature indication, results of prior testing procedures, meal and/or exercise indicators, and/or various combinations thereof. The instrument 10 may also include a navigation button 42, a scroll wheel, or any mechanism suitable for scrolling through the information displayed on the display 16.

At least one of the buttons 22a, 22b, 42 may be depressed to operate the electronics of the instrument 10. The instrument 10 typically includes a microprocessor or the like for processing and/or storing data generated during the testing procedure. It is contemplated that the number of buttons on the instrument may be different than depicted in FIGS. 1a and 3a,b. It is contemplated that the number of buttons may be increased to provide the user with a means to input notes into the memory. For example, the user may input a note into the memory that he or she had exercised, eaten, or taken a medication prior to testing. Thus, the user may store an electronic log along with a glucose reading history within the instrument 10. The buttons may also be used to set and display date and time information and to activate alarms that remind the user to, for example, conduct a blood glucose test according to a predetermined schedule. The buttons may also be used to activate certain calibration procedures for the instrument 10. It may be desirable for the buttons to be large to assist those individuals with poor hand/finger coordination and/or poor vision. For example, the buttons may have a diameter of at least 0.25 inch. It is contemplated that the buttons may be shaped differently than those shown in FIGS. 1a and 3a,b.

According to another embodiment, the display 16 may communicate in an audible manner instead of or in addition to the above-discussed visual manner. Thus, the display 16 may be designed to operate in audible and visual manners.

The instrument 10 includes at least one compartment 18a,b that is adapted to contain a lancing device. Turning to FIGS. 2a,b, a manually-operated lancing device 100 for obtaining a fluid sample from a test subject is illustrated, according to one embodiment. The lancing device 100 has a main housing 120 and a movable housing 114 that is movable relative to the main housing 120. An endcap support 160 is connected to the main housing 120 on the testing end of the lancing device 100.

An endcap 180 may be removably attached to the endcap support 160. When attached, the endcap 180 is retained on the endcap support 160 by a pair of support arms 190a-b integrally formed with the endcap support 160.

To use the lancing device 100, the movable housing 114 is pulled away from the main housing 120 to move an internal lancet mechanism 129 to a cocked position, and then a pushbutton 122 is pushed to actuate the lancet mechanism 129 so that a sharp tip 131 of a lancet is forced through an aperture in the endcap 180. The lancing device 100 may be provided with a number of different endcaps 180, each having a different opening, diameter, and/or shape, to facilitate the formation of skin punctures of various depths. Alternatively, the endcap 180 may include an adjustable dial 124 for allowing punctures of different depths to be performed utilizing a single endcap 180. It is contemplated that other types of lancet devices may be also used with the instrument 10.

According to another embodiment, a lancing device is attached to and/or incorporated within the first portion, as shown in FIGS. 3a, 3b. The lancing device 214 of this embodiment includes a lancet holder 230 (see FIG. 3b) and a lancet cover 232 (see FIG. 3a). The lancet holder is adapted to hold a lancet 234 as shown, for example, in FIG. 3b. It is contemplated that the instrument may include an eject mechanism 264 that is adapted to eject at least the lancet 234.

According to one embodiment, the lancing device 214 is manually operated. If the lancet device 214 is manually operated, the lancet device 214 according to one process is cocked by the user and then activated by pressing a button. For example, a button 240 may be used to activate a lancet 234 in a manually operated lancet device 214. It is further contemplated that the lancet 234 may be activated by techniques other than pressing the button 240. For example, the lancet 234 may be activated by a lever mechanism. It is farther contemplated that the manually operated lancet device 214 may further include a mains-powered vacuum to assist in enhancing the fluid flow.

Alternatively, the lancing device 214 of FIGS. 3a, 3b may be electronically operated. For example, the lancet 234 may be activated by pressing the button 240. From a user's standpoint, the process of activating the lancet 234 is simplified when the lancing device 214 is electronically operated. It is contemplated that the electronically operated lancet device 214 may further include a mains-powered vacuum to assist in enhancing the fluid flow.

Referring back to FIG. 1, if electrochemical test sensors are used in the instrument 10, then one of the test sensors will be properly aligned with one or more electrical contacts housed within the instrument 10. The testing end of the sensor then receives, for example, a drop of blood to be tested, whereby the blood is analyzed by an electrochemical circuit. The results of the analysis are then displayed on the display 16 of the instrument 10. It is contemplated that other types of sensors may be used, such as optical sensors.

A test sensor may be removed from, for example, one of the compartments 18a,b and then manually placed in the test-sensor opening 20. According to another process, a test sensor may be automatically advanced to the opening 20 by utilizing the user-interface (e.g., a user pressing one of the buttons 22a,b). The instrument 10 is generally turned on after the test sensor is placed into or advanced into the test-sensor opening 20. After the instrument 10 is powered on, the testing is ready to begin.

The user typically places his/her finger up to a lancing device to generate a whole blood sample. It is contemplated that a blood sample may be generated from other areas of the body. The user then removes his/her finger from the lancing device and brings the whole blood sample into contact with the sensor, wherein the blood is generally drawn into the sensor by capillary action. The test sensors are typically provided with a capillary channel that extends from the front or testing end of the sensors to biosensing or reagent material disposed in the sensor. The biosensing or reagent material is designed to react with the desired analyte to be tested. When the testing end of the sensor is placed into fluid (e.g., blood that is accumulated on a person's finger after the finger has been pricked), a portion of the fluid is drawn into the capillary channel by capillary action. The fluid then chemically reacts with the reagent material in the sensor so that an electrical signal indicative of the blood glucose level being tested is supplied and subsequently transmitted to an electrical assembly. After a minimum amount of blood is drawn into the test sensor, the testing is performed, and the result is, for example, shown on the display 16 and stored in memory. The result of the testing may also be announced audibly, by, for example, using a speaker, and stored in memory.

After the testing has been completed, the test sensor may be removed from the test-sensor opening 20 by several methods. In one embodiment, the instrument 10 may include an eject mechanism 40 that ejects the used test sensor from the instrument 10. Such an eject mechanism 40 may automatically move the used test sensor into one of the compartments 18a,b. In such an embodiment, the test sensors are released forcefully. In a further embodiment, the test sensor may be removed manually from the instrument 10.

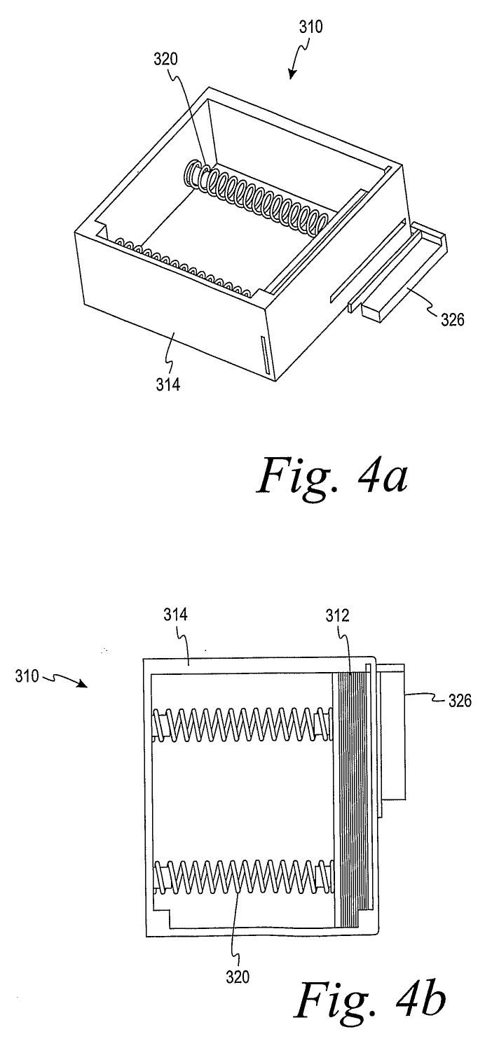

According to another embodiment, a disposable cartridge that contains a plurality of test sensors may be used. One example of a disposable cartridge that may be used in the instrument 10 is depicted in FIGS. 4a, 4b. The disposable cartridge 310 of FIGS. 4a, 4b comprises a housing 314, a plurality of stacked test sensors 312, a movable mechanism 326, a mechanical mechanism 320, and a plurality of moveable seals (not shown). The cartridge 310 is adapted to be disposable after each of the plurality of test sensors 312 has been used. After each of the plurality of test sensors 312 has been used, the cartridge 310 may be removed from the instrument 10 and replaced with a second identical cartridge that includes a plurality of unused test sensors.

The instrument 10 according to a further embodiment may include a programmable alarm 44 to alert the user to begin testing. The alarm 44 is programmed to sound at a predetermined schedule. An alarm is especially useful for those individuals who have poor memory as well as those individuals who become easily preoccupied and/or forget to test according to a predetermined schedule.

The instrument 10 may include a built-in data management system 46 that is accessible to remote monitoring by, for example, a physician. Such a built-in data management system 46 may be connected for remote monitoring by, for example, a telephone line 48 and a modem 50.

The instrument 10 may also include a bar code reader that reads a bar code label on a disposable test-sensor cartridge. The bar code reader may determine information such as the lot number and calibration numbers for a particular test sensor being used.

### Alternative Embodiment A

An integrated-diagnostic instrument adapted to determine an analyte concentration of a fluid sample using a test sensor, the instrument comprising:
a first portion including at least one opening formed therein, the opening being adapted to receive a test sensor, the first portion forming at least one compartment adapted to receive a lancing device and including a user-interface mechanism; and
a second portion being hingedly connected to the first portion, the second portion including a display.

### Alternative Embodiment B

The instrument of Alternative Embodiment A, wherein the at least one compartment includes a lancing device.

### Alternative Embodiment C

The instrument of Alternative Embodiment B, wherein the lancing device is adapted to be manually operated.

### Alternative Embodiment D

The instrument of Alternative Embodiment B, wherein the lancing device is adapted to be electronically operated.

### Alternative Embodiment E

The instrument of Alternative Embodiment B, wherein the lancing device is coupled to the first portion.

### Alternative Embodiment F

The instrument of Alternative Embodiment B, wherein the lancing device includes a vacuum to assist in enhancing the fluid sample flow.

### Alternative Embodiment G

The instrument of Alternative Embodiment A, wherein the instrument has a footprint of less than 12 in².

### Alternative Embodiment H

The instrument of Alternative Embodiment A, wherein the display has an area of at least 6 in².

### Alternative Embodiment I

The instrument of Alternative Embodiment A, wherein the instrument communicates the analyte concentration to a user in an audible manner.

### Alternative Embodiment J

The instrument of Alternative Embodiment A, wherein the first portion or second portion further includes a programmable alarm to alert a user to test at predetermined intervals.

### Alternative Embodiment K

The instrument of Alternative Embodiment A, wherein the first portion or second portion further includes a modem.

### Alternative Embodiment L

The instrument of Alternative Embodiment A, wherein the at least one compartment is a plurality of compartments.

### Alternative Embodiment M

The instrument of Alternative Embodiment A, wherein the first portion further includes an eject mechanism that is adapted to eject the test sensor.

### Alternative Embodiment N

The instrument of Alternative Embodiment A, wherein the first portion further includes an eject mechanism that is adapted to eject the lancet.

### Alternative Embodiment O

The instrument of Alternative Embodiment A, wherein the first portion and second portion are adapted to releasably lock therewith.

### Alternative Embodiment P

The instrument of Alternative Embodiment A, wherein the user-interface mechanism is a plurality of buttons.

### Alternative Embodiment Q

The instrument of Alternative Embodiment A, wherein the at least one compartment includes a detachable cover.

### Alternative Embodiment R

An integrated-diagnostic instrument adapted to determine an analyte concentration of a fluid sample using a test sensor, the instrument comprising:
a first portion including a user-interface mechanism, the first portion including at least one opening formed therein, the opening being adapted to receive a test sensor, the first portion forming a plurality of compartments, a first compartment including a plurality of test sensors, a second compartment including a lancing device; and
a second portion being hingedly connected to the first portion, the second portion including a display.

### Alternative Embodiment S

The instrument of Alternative Embodiment R, wherein the lancing device is adapted to be manually operated.

### Alternative Embodiment T

The instrument of Alternative Embodiment R, wherein the lancing device is adapted to be electronically operated.

### Alternative Embodiment U

The instrument of Alternative Embodiment R, wherein the lancing device is coupled to the first portion.

### Alternative Embodiment V

The instrument of Alternative Embodiment R, wherein the lancing device includes a vacuum to assist in enhancing the fluid sample flow.

### Alternative Embodiment W

The instrument of Alternative Embodiment R, wherein the display has an area of at least 3.75 in².

### Alternative Embodiment X

The instrument of Alternative Embodiment R, wherein the instrument communicates the analyte concentration to a user in an audible manner.

### Alternative Embodiment Y

The instrument of Alternative Embodiment R, wherein the first portion or second portion further includes a programmable alarm to alert a user to test at predetermined intervals.

### Alternative Embodiment Z

The instrument of Alternative Embodiment R, wherein the first portion or second portion further includes a modem.

### Alternative Embodiment AA

The instrument of Alternative Embodiment R, wherein the first portion further includes an eject mechanism that is adapted to eject the test sensor.

### Alternative Embodiment AB

The instrument of Alternative Embodiment R, wherein the first portion further includes an eject mechanism that is adapted to eject the lancet.

### Alternative Embodiment AC

The instrument of Alternative Embodiment R, wherein the first portion and second portion are adapted to releasably lock therewith.

### Alternative Embodiment AD

The instrument of Alternative Embodiment R, wherein the at least one user-interface mechanism is a plurality of buttons.

### Alternative Embodiment AE

The instrument of Alternative Embodiment R, wherein the first and second compartments include a detachable cover.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents, and alternatives falling within the spirit and scope of the invention as defined by the appended claims.

1. An integrated-diagnostic instrument adapted to determine an analyte concentration of a fluid sample using a test sensor, the instrument comprising:
a first portion including at least one opening formed therein, the opening being adapted to receive a test sensor, the first portion forming at least one compartment adapted to receive a lancing device and including a user-interface mechanism; and
a second portion being hingedly connected to the first portion, the second portion including a display.

2. The instrument of claim 1, wherein the at least one compartment includes a lancing device.

3. The instrument of claim 2, wherein the lancing device is adapted to be manually operated.

4. The instrument of claim 2, wherein the lancing device is adapted to be electronically operated.

5. The instrument of claim 2, wherein the lancing device is coupled to the first portion.

6. The instrument of claim 2, wherein the lancing device includes a vacuum to assist in enhancing the fluid sample flow.

7. The instrument of claim 1, wherein the instrument has a footprint of less than 12 in².

8. The instrument of claim 1, wherein the display has an area of at least 6 in².

9. The instrument of claim 1, wherein the instrument communicates the analyte concentration to a user in an audible manner.

10. The instrument of claim 1, wherein the first portion or second portion further includes a programmable alarm to alert a user to test at predetermined intervals.

11. The instrument of claim 1, wherein the first portion or second portion further includes a modem.

12. (canceled)

13. The instrument of claim 1, wherein the first portion further includes an eject mechanism that is adapted to eject the test sensor or the lancet.

14. (canceled)

15. The instrument of claim 1, wherein the first portion and second portion are adapted to releasably lock therewith.

16. (canceled)

17. The instrument of claim 1, wherein the at least one compartment includes a detachable cover.

18. An integrated-diagnostic instrument adapted to determine an analyte concentration of a fluid sample using a test sensor, the instrument comprising:
a first portion including a user-interface mechanism, the first portion including at least one opening formed therein, the opening being adapted to receive a test sensor, the first portion forming a plurality of compartments, a first compartment including a plurality of test sensors, a second compartment including a lancing device; and
a second portion being hingedly connected to the first portion, the second portion including a display.

19. The instrument of claim 18, wherein the lancing device is adapted to be manually operated.

20. The instrument of claim 18, wherein the lancing device is adapted to be electronically operated.

21. The instrument of claim 18, wherein the lancing device is coupled to the first portion.

22. The instrument of claim 18, wherein the lancing device includes a vacuum to assist in enhancing the fluid sample flow.

23. The instrument of claim 18, wherein the display has an area of at least 3.75 in².

24. The instrument of claim 18, wherein the instrument communicates the analyte concentration to a user in an audible manner.

25. The instrument of claim 18, wherein the first portion or second portion further includes a programmable alarm to alert a user to test at predetermined intervals.

26. The instrument of claim 18, wherein the first portion or second portion further includes a modem.

27. The instrument of claim 18, wherein the first portion further includes an eject mechanism that is adapted to eject the test sensor or the lancet.

28. (canceled)

29. (canceled)

30. (canceled)

31. (canceled)
